(19) United States
(12) Patent Application Publication
(10) Pub. No.: US 2019/0224097 A1
(43) Pub. Date: Jul. 25, 2019

(54) IMPROVED CONDITIONING HAIR TREATMENT PRODUCT WITH WASHOUT PROTECTION

(71) Applicant: Henkel AG & Co. KGaA, Duessekdorf (DE)

(72) Inventors: VOLKER SCHEUNEMANN, Lueneburg (DE); ERIK SCHULZE ZUR WIESCHE, Hamburg (DE); RENE KROHN, Norderstedt (DE)

(73) Assignee: Henkel AG & Co. KGaA, Duesseldorf (DE)

(21) Appl. No.: 16/337,302

(22) PCT Filed: Jul. 24, 2017
(86) PCT No.: PCT/EP2017/068583

§ 371 Date: Mar. 27, 2019

(30) Foreign Application Priority Data
Sep. 30, 2016 (DE) 10 2016 219 007.5

(51) Int. Cl. A61K 8/49; A61K 8/64; A61K 8/41; A61K 8/34; A61K 8/33; A61K 8/73; A61K 8/65; A61K 8/44; A61Q 5/12; A61Q 5/02

(52) U.S. Cl. CPC A61K 8/4913; A61K 8/64; A61K 8/416; A61K 8/345; A61K 8/33; A61K 8/34; A61Q 5/02; A61K 8/737; A61K 8/65; A61K 8/44; A61K 8/731; A61Q 5/12; A61K 8/645

(57) ABSTRACT

Hair treatment agents and methods for treating hair are provided. An exemplary hair treatment agent includes, based on the weight of the hair treatment agent, from about 0.001 to about 10 wt. % of at least one succinimidyl ester, and from about 0.001 to about 20 wt. % of at least one protein hydrolyzate.

### CROSS-REFERENCE TO RELATED APPLICATION

This application is a U.S. National-Stage entry under 35 U.S.C. § 371 based on International Application No. PCT/EP2017/068583, filed Jul. 24, 2017, which was published under PCT Article 21(2) and which claims priority to German Application No. 10 2016 219 007.5, filed Sep. 30, 2016, which are all hereby incorporated in their entirety by reference.

### TECHNICAL FIELD

The present disclosure relates to hair treatment agents, in particular shampoos and what are referred to as conditioners, having an active ingredient combination for the gentle and effective care of hair.

### BACKGROUND

The importance of care products with the longest-lasting possible effect is increasing not least due to the high amount of strain on hair caused, for example, by coloring or permanents, by cleaning hair with shampoos, and by environmental pressures. Care products of this type influence the natural structure and properties of hair. For example, the wet and dry combability, the hold and the volume of hair can be optimized following care treatments of this kind, or hair can be protected from increased splitting.

It has long been customary, therefore, to subject hair to a special aftertreatment. In this process, the hair is treated with special active ingredients, for example quaternary ammonium salts or special polymers, usually in the form of a rinse. As a result of this treatment, depending on the formulation, the combability, the hold, and the volume of the hair are improved, and the amount of splitting is reduced.

Multi-functional cosmetic products are also known in the prior art. These include in particular what are referred to as “2 in 1” shampoos, which not only clean hair but also condition it. Products of this type are held in high esteem by consumers because, due to their product performance, they eradicate the need for at least one process step, for example conditioning with a conventional hair conditioner.

Similarly, products for modifying natural hair color play a prominent role in hair cosmetics. A distinction is made between permanent, semi-permanent, or temporary coloring systems based on chemical and/or natural dyes. Hair colors produced artificially by permanent, semi-permanent or temporary coloring systems are, however, disadvantageous in that they can change undesirably, for example during or after hair cleaning.

An “undesirable change” is understood to mean fading or bleeding and loss of the color brilliance of the hair shade achieved by the particular coloring. Environmental impacts and/or the effects of sunlight can further intensify these changes.

There continues to be a need to provide active ingredients or active ingredient combinations for hair treatment agents which have good nourishing properties and also strengthen the adhesion of dyes to hair fibers and thus maintain the fastness of the artificially produced hair color, and in this respect to develop hair treatment agents.

It has been observed, however, that water hardness can sometimes negatively influence the nourishing properties of hair treatment agents, and therefore the nourishing performance of the same product may be either too low or too high depending on water quality.

Nourishing performance that is too high or “over-nourishment” (occurrence of what is known as a build-up effect) of the hair is understood to mean in particular a greasy hair feel, lack of hair volume, and/or a lank, unkempt appearance of the hair, whereas damaged hair (broken and dull hair that is prone to splitting) is an indicator of too little nourishing performance.

### BRIEF SUMMARY

Hair treatment agents and methods for treating hair are provided. An exemplary hair treatment agent includes, based on the weight of the hair treatment agent, from about 0.001 to about 10 wt. % of at least one succinimidyl ester, and from about 0.001 to about 20 wt. % of at least one protein hydrolyzate.

In another embodiment, a method for treating hair includes applying a hair treatment agent to dry or wet hair, wherein the hair treatment agent comprises, based on the weight of the hair treatment agent, from about 0.001 to about 10 wt. % of at least one succinimidyl ester, and from about 0.001 to about 20 wt. % of at least one protein hydrolysate. The method further includes leaving the hair treatment agent on the hair for a selected period of time, and subsequently rinsing the hair treatment agent from the hair.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present application was therefore to provide skin-compatible, nourishing hair treatment agents which have excellent foaming properties, can be rinsed out well with water, and additionally reduce or prevent washout of color from colored hair.

The nourishing hair treatment agents should have a uniform conditioning performance irrespective of water quality and in particular should not weigh down fine hair and/or damaged hair, but should strengthen the structure of the hair and thus protect it from splitting and breaking and should improve combability and the feel of the hair. In addition, said agents should have a germicidal effect.

It has now been found that a combination of certain ingredients has a particularly positive effect on colored hair and the hair follicles treated with said ingredients.

A first subject of the present disclosure is hair treatment agents containing, based on their weight,

a) from about 0.001 to about 10 wt. % of at least one succinimidyl ester,

b) from about 0.001 to about 20 wt. % of at least one protein hydrolyzate.

Hair treatment agents within the context of the present disclosure are, for example, hair shampoos, hair conditioners, conditioning shampoos, hair sprays, hair rinses, hair masques, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, agents that men use already are preferred. Preferred agents are therefore shampoos, conditioning agents, or hair tonics.

The hair treatment agents contain, as a first essential ingredient, from about 0.001 to about 10 wt. % of at least one succinimidyl ester.

Succinimidyl esters are esters of carboxylic acids having (optionally substituted) N-hydroxysuccinimide (NHS, IUPAC 1-hydroxy-2,5-pyrrolidinedione) and are also referred to as NHS esters. Hair treatment agents which are preferred as contemplated herein additionally contain, based on the weight of the agent, from about 0.01 to about 10 wt. %, preferably from about 0.05 to about 7.5 wt. %, more preferably from about 0.1 to about 6 wt. %, and in particular from about 0.15 to about 5 wt. %, of (a) succinimidyl ester(s) of formula (III)

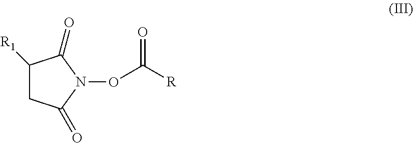

in which
R1 represents —H or an ionic group,
R represents an optionally substituted saturated or unsaturated, linear, branched or cyclic, aliphatic or aromatic hydrocarbon functional group having at least 5 C atoms.

Suitable ionic groups R1 include both anionic groups such as phosphate, phosphonate, phosphinate, sulfate, sulfonate, sulfinate, sulfenate, oxysulfonate, carboxylate groups and cationic groups such as substituted or unsubstituted ammonium groups. Zwitterionic/betainic groups such as carboxybetaine or sulfobetaine groups are also possible.

In succinimidyl esters which are preferred as contemplated herein, R1 represents —H, —OSO₃⁻, —SO₃⁻, —SO₂⁻, —COO⁻, —NH₃⁺, —N(CH₃)H₂⁺, —N(CH₃)₂H⁺, —N(CH₃)₃⁺, —N+(CH₃)₂(CH₂)₂—COO⁻, or —N⁺(CH₃)₂(CH₂)₃—SO₃⁻.

Succinimidyl esters that are preferably to be used as contemplated herein are described on pages 4 to 6 of the priority document as numbers 1 to 72.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one succinimidyl ester from the group

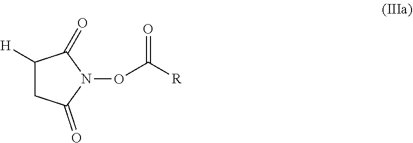

in which R represents —Ph or —(CH₂)nCH₃, where n=4, 5, 6, 7, 8, 9 or 10,

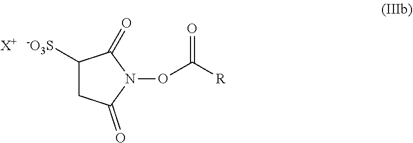

in which R represents —Ph or —(CH₂)nCH₃, where n=4, 5, 6, 7, 8, 9 or 10, and

X represents H, a monovalent cation, or the n-th part of an n-valent cation,

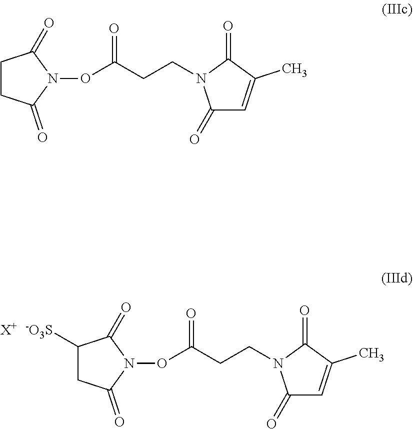

Hair treatment agents containing these succinimidyl esters have excellent nourishing properties on both thin and normal hair, bring about significant structure strengthening, and prevent washout and bleeding of chemically colored hair very effectively.

As further essential components, the agents as contemplated herein contain from about 0.001 to about 20 wt. % of at least one protein hydrolyzate.

As contemplated herein, protein hydrolyzates are degradation products of proteins which are prepared by acidic, basic or enzymatic reaction. Due to the preparation process, protein hydrolyzates have a distributed molecular weight. The protein hydrolyzates as contemplated herein also include oligopeptides, as these can also be produced from proteins by corresponding reactions. As contemplated herein, the protein hydrolyzates within the meaning of this present disclosure therefore do not include single amino acids, which are present as a discrete individual compound.

Protein hydrolyzates of plant, animal, marine or synthetic origin can be used as contemplated herein.

Animal protein hydrolyzates are, for example, elastin, collagen, keratin, silk, and milk protein hydrolyzates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), ProSina® (Croda) and Kerasol® (Croda).

Furthermore, plant protein hydrolyzates such as soybean, almond, pea, moringa, potato, and wheat protein hydrolyzates are preferred as contemplated herein. Such products are available, for example, under the trademarks Gluadin® (Cognis), DiaMin® (Diamalt), Lexein® (Inolex), Hydrosoy ® (Croda), Hydrolupin® (Croda), Hydrosesame® (Croda), Hydrotitium® (Croda), Crotein® (Croda) and Puricare® LS 9658 from the company Laboratoires Sérobiologiques.

Further protein hydrolyzates which are preferred as contemplated herein are of marine origin. This includes, for example, collagen hydrolyzates from fish or algae and protein hydrolyzates from mussels or pearl hydrolyzates. Examples of pearl extracts as contemplated herein are the trade products Pearl Protein Extract BG® or Crodarom® Pearl.

Furthermore, protein hydrolyzates and derivatives thereof include cationized protein hydrolyzates, it being possible for the underlying protein hydrolyzate to originate from animals, for example from collagen, milk, or keratin, from plants, for example from wheat, corn, rice, potatoes, soybeans, or almonds, from marine life forms, for example from fish collagen or algae, or from biotechnologically obtained protein hydrolyzates. Typical examples of cationic protein hydrolyzates and derivatives as contemplated herein are the commercially available products known under the INCI names in the “International Cosmetic Ingredient Dictionary and Handbook” (seventh edition 1997, The Cosmetic, Toiletry, and Fragrance Association 1101 17^{th} Street, N.W., Suite 300, Washington, D.C. 20036-4702).

Hair treatment agents which are preferred as contemplated herein contain, based on their weight, from about 0.01 to about 15 wt. %, preferably from about 0.05 to about 10 wt. %, more preferably from about 0.1 to about 7.5 wt. %, and in particular from about 0.15 to about 5 wt. %, protein hydrolyzate(s) from the group elastin, collagen, keratin, silk, and milk protein hydrolyzate, soybean, almond, pea, moringa, potato, and wheat protein hydrolyzate.

Oligopeptides are also protein hydrolyzates as contemplated herein. Oligopeptides may be preferred in the hair treatment agents as contemplated herein owing to their defined amino acid sequence.

An oligopeptide which has at least one amino acid sequence Glu-Glu-Glu, it being possible for the amino group to be present in a free or protonated form and for the carboxyl groups to be present in a free or deprotonated form, may be particularly preferred as contemplated herein.

In this formula, as in all preceding formulas, the bracketed hydrogen atom of the amino group, and the bracketed hydroxyl group of the acid function, means that the groups in question can be present as such (then, it is an oligopeptide having the relevant number of amino acids as in the preceding formula), or that the amino acid sequence is present in an oligopeptide which also comprises further amino acids; depending on where the further amino acid(s) is/are bonded, the bracketed constituents of the above-mentioned formula are replaced by the further amino acid functional group(s).

Within the context of the present application, oligopeptides are condensation products of amino acids which are linked by peptide bonds in the manner of an acid amide and which comprise at least about 3 and at most about 25 amino acids. In hair treatment agents which are preferred as contemplated herein, the oligopeptide comprises from about 5 to about 15 amino acids, preferably from about 6 to about 13 amino acids, particularly preferably from about 7 to about 12 amino acids, and in particular about 8, about 9 or about 10 amino acids. Depending on whether further amino acids are bound to the sequence Glu-Glu-Glu and depending on the type of said amino acids, the molar mass of the oligopeptide contained in the agents as contemplated herein may vary. Hair treatment agents which are preferred as contemplated herein are exemplified in that the oligopeptide has a molar mass of from about 650 to about 3000 daltons, preferably from about 750 to about 2500 daltons, particularly preferably from about 850 to about 2000 daltons, and in particular from about 1000 to about 1600 daltons. As can be seen from the preferred number of amino acids in the oligopeptides and the preferred molar mass range, oligopeptides are preferably used which not only consist of the three glutamic acids but also have additional amino acids bound to this sequence. These further amino acids are preferably selected from certain amino acids, whereas certain other representatives are less preferred as contemplated herein. A particularly preferred oligopeptide additionally contains tyrosine, which is preferably bound via its acid function to the sequence Glu-Glu-Glu. Hair treatment agents which are preferred as contemplated herein are therefore exemplified in that the oligopeptide contained therein comprises at least one amino acid sequence Tyr-Glu-Glu-Glu, it being possible for the amino group to be present in a free or protonated form and for the carboxy groups to be present in a free or deprotonated form.

A further particularly preferred oligopeptide additionally contains isoleucine, which is preferably bound via its amino function to the sequence Glu-Glu-Glu. Hair treatment agents which are preferred as contemplated herein are therefore exemplified in that the oligopeptide contained therein comprises at least one amino acid sequence Glu-Glu-Glu-Ile, it being possible for the amino group to be present in a free or protonated form and for the carboxy groups to be present in a free or deprotonated form.

Oligopeptides that have both of the aforementioned amino acids (tyrosine and isoleucine) are preferred as contemplated herein. In this case, hair treatment agents as contemplated herein are particularly preferred in which the oligopeptide contained therein has at least one amino acid sequence Tyr-Glu-Glu-Glu-Ile, it being possible for the amino group to be present in a free or protonated form and for the carboxy groups to be present in a free or deprotonated form.

Further preferred oligopeptides additionally contain arginine, which is preferably present bound to isoleucine.

Still further preferred oligopeptides additionally contain valine, which is preferably present bound to arginine. Hair treatment agents which are further preferred as contemplated herein are therefore exemplified in that the oligopeptide contained therein comprises at least one amino acid sequence Tyr-Glu-Glu-Glu-Ile-Arg-Val, it being possible for the amino group to be present in a free or protonated form and for the carboxy groups to be present in a free or deprotonated form.

Still further preferred oligopeptides additionally contain leucine, which is preferably present bound to valine. Hair treatment agents which are further preferred as contemplated herein are exemplified in that the oligopeptide contained therein comprises at least one amino acid sequence Tyr-Glu-Glu-Glu-Ile-Arg-Val-Leu, it being possible for the amino group to be present in a free or protonated form and for the carboxy groups to be present in a free or deprotonated form.

Particularly preferred oligopeptides additionally contain leucine, which is preferably present bound to tyrosine. Hair treatment agents which are further preferred as contemplated herein are exemplified in that the oligopeptide contained therein comprises at least one amino acid sequence Leu-Tyr-Glu-Glu-Glu-Ile-Arg-Val-Leu, it being possible for the amino group to be present in a free or protonated form and for the carboxy groups to be present in a free or deprotonated form.

Protein hydrolyzates which are particularly preferred as contemplated herein are protein hydrolyzates based on keratin, silk, wheat, and moringa, and marine protein hydrolyzates. Protein hydrolyzates based on silk, keratin, and wheat are most preferred.

The protein hydrolyzates are contained in the compositions in concentrations of from about 0.001 wt. % to about 20 wt. %, preferably from about 0.05 wt. % to about 15 wt. %, and very particularly preferably in amounts of from about 0.05 wt. % to about 5 wt. %.

The agents as contemplated herein may contain amino-functional silicones to further increase the conditioning effects. Preferred agents as contemplated herein contain from about 0.001 to about 50 wt. % of at least one amino-functional silicone.

Particularly preferred agents as contemplated herein contain at least one amino-functional silicone of formula (Si-IIa)

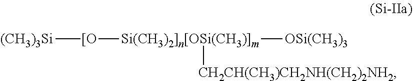

in which m and n are numbers whose sum (m+n) is between about 1 and about 2000, preferably between about 50 and about 150, n preferably assuming values of from 0 to about 1999 and in particular from about 49 to about 149 and m preferably assuming values of from about 1 to about 2000, in particular from about 1 to about 10.

These silicones are referred to as trimethylsilylamodimethicone, in accordance with the INCI declaration.

Particularly preferred as well are agents as contemplated herein that contain an amino-functional silicone of formula (Si-IIb)

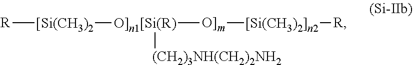

in which R represents —OH, —O—CH₃, or a —CH₃ group and m, n1 and n2 are numbers whose sum (m+n1+n2) is between about 1 and about 2000, preferably between about 50 and about 150, the sum (n1+n2) preferably assuming values of from 0 to about 1999 and in particular from about 49 to about 149 and m preferably assuming values of from about 1 to about 2000, in particular from about 1 to about 10.

These silicones are referred to as amodimethicone, in accordance with the INCI declaration.

Irrespective of which amino-functional silicones are used, agents as contemplated herein are preferred that contain an amino-functional silicone of which the amine value is above about 0.25 meq/g, preferably above about 0.3 meq/g, and in particular above about 0.4 meq/g. The amine value here represents the milliequivalents of amine per gram of the amino-functional silicone. Said value can be determined by titration and may also be given in the unit mg KOH/g.

Hair treatment agents which are preferred as contemplated herein contain, based on their weight, from about 0.01 to about 20 wt. %, preferably from about 0.05 to about 10 wt. %, more preferably from about 0.1 to about 7.5 wt. %, and in particular from about 0.15 to about 5 wt. %, amino-functional silicone(s).

The agents as contemplated herein particularly preferably contain amino-functional silicone(s) having terminal hydroxy group(s). Some special amino-functional silicone(s) having terminal hydroxy group(s) have been found to be particularly suitable in the agents as contemplated herein. These are described in the following.

Pretreatment agents that contain at least one silicone of formula (Si-V) have been found to be particularly effective in the method as contemplated herein with regard to the desired effects:

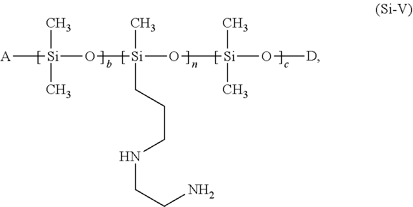

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and about 1000,
   with the proviso that
   n>0 and b+c>0
   at least one of the conditions A=—OH or D=—H is met.

Hair treatment agents as contemplated herein that contain, based on their weight, from about 0.01 to about 20 wt. %, preferably from about 0.01 to about 20 wt. %, more preferably from about 0.1 to about 10 wt. %, even more preferably from about 0.5 to about 7.5 wt. %, and in particular from about 0.1 to about 5 wt. %, of at least one silicone of formula (Si-V):

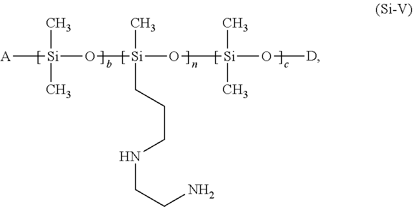

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and about 1000,
   with the proviso that
   n>0 and b+c>0
   at least one of the conditions A=—OH or D=—H is met,
      are therefore preferred as contemplated herein.

In the above formula (Si-V), the individual siloxane units having the indices b, c and n are randomly distributed, i.e. they are not necessarily block copolymers.

Further particularly suitable silicones are 4-morpholinomethyl-substituted. Hair treatment agents as contemplated herein that contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone which comprises structural units of formulas (Si-VIa), (Si-VIb) and (Si-VIc)

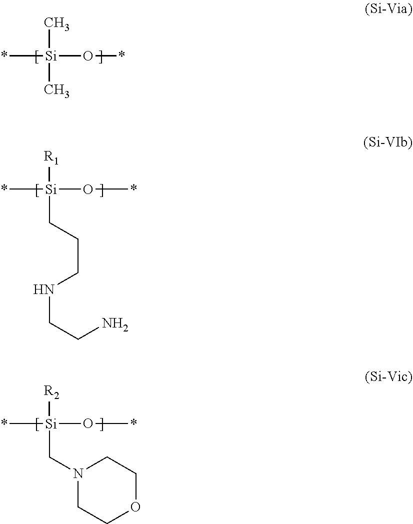

in which
R1 represents —CH₃, —OH, —OCH₃, —O—CH₂CH₃, —O—CH₂CH₂CH₃, or —O—CH(CH₃)₂,
R2 represents —CH₃, —OH, or —OCH₃,
   are particularly preferred.

Particularly preferred hair treatment agents as contemplated herein contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone of formula (Si-VI)

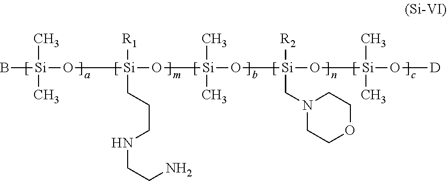

in which
R1 represents —CH₃, —OH, —OCH₃, —O—CH₂CH₃, —O—CH₂CH₂CH₃, or —O—CH(CH₃)₂,
R2 represents —CH₃, —OH, or —OCH₃,
B represents an —OH, —O—Si(CH₃)₃, —O—Si(CH₃)₂OH, or —O—Si(CH₃)2OCH₃ group,
D represents an —H, —Si(CH₃)₃, —Si(CH₃)₂OH, or —Si(CH₃)₂OCH₃ group,
a, b and c represent, independently of one another, integers between 0 and about 1000, with the
proviso that a+b+c>0;
m and n represent, independently of one another, integers between about 1 and about 1000,
with the proviso that
   at least one of the conditions B=—OH or D=—H is met,
   the units a, b, c, m and n are distributed randomly or in blocks in the molecule.

Structural formula (Si-VI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m and n are preferably distributed randomly.

The silicones represented by formula (Si-VI) and used as contemplated herein can be trimethylsilyl-terminated (D or B=—Si(CH₃)₃), but they may also be dimethylsilylhydroxy -terminated at both ends or dimethylsilylhydroxy-terminated at one end and dimethylsilylmethoxy-terminated at the other end. Within the context of the present disclosure, silicones which are particularly preferably used are selected from silicones in which

B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.

These silicones lead to enormous improvements in the hair properties of hair treated with the agents as contemplated herein, and to greatly improved protection during oxidative treatment.

Irrespective of the type of amino-functional silicone(s) having terminal hydroxy group(s) used, the agents as contemplated herein contain the silicone(s) preferably in the form of an emulsion, particularly preferably in the form of a microemulsion.

It has been found that the effect of the silicones used in the agents as contemplated herein can be increased further still if certain non-ionic components are also used in the agents. In addition, these nonionic components have positive effects on the storage stability of the agents. Nonionic components that are particularly suitable here are ethoxylates of decanol, undecanol, dodecanol, tridecanol, etc. Ethoxylated tridecanols which are particularly preferably incorporated in the agents as contemplated herein have been found to be particularly suitable. Agents which are particularly preferred as contemplated herein contain, based on their weight, from about 0.00001 to about 5 wt. %, preferably from about 0.0001 to about 3.5 wt. %, particularly preferably from about 0.001 to about 2 wt. %, more preferably from about 0.01 to about 1 wt. %, and in particular from about 0.1 to about 0.5 wt. %, branched ethoxylated tridecanol (INCI name: trideceth-5) or α-iso-tridecyl-ω-hydroxy polyglycol ether (INCI name: trideceth-10) or mixtures thereof.

In addition to or instead of the amino-functional silicone(s), the agents as contemplated herein may contain further silicone(s) which is/are not amino-functional.

Hair treatment agents which are preferred as contemplated herein contain, based on their weight, from about 0.01 to about 20 wt. %, preferably from about 0.1 to about 10 wt. %, more preferably from about 0.5 to about 7.5 wt. %, and in particular from about 1 to about 5 wt. %, non-amino-functional silicone(s).

Preferred silicones are described in the following.

Particularly preferred agents as contemplated herein contain at least one silicone of formula Si-I

(CH₃)₃Si—[O—Si(CH₃)₂]ₓ—O—Si(CH₃)₃ (Si-I),

in which x represents a number from 0 to about 100, preferably from 0 to about 50, more preferably from 0 to about 20, and in particular from 0 to about 10.

These silicones are referred to as dimethicone, in accordance with INCI nomenclature. The following compounds are preferably used within the context of the present disclosure as a silicone of formula Si-I:

(CH₃)₃Si—O—Si(CH₃)₃ (CH₃)₃Si—O—(CH₃)₂Si—O—Si(CH₃)₃ (CH₃)₃Si—[O—(CH₃)₂Si]₂—O—Si(CH₃)₃
(CH₃)₃Si—[O—(CH₃)₂Si]₃—O—Si(CH₃)₃ (CH₃)₃Si—[O—(CH₃)₂Si]₄—O—Si(CH₃)₃
(CH₃)₃Si—[O—(CH₃)₂Si]₅—O—Si(CH₃)₃ (CH₃)₃Si—[O—(CH₃)₂Si]₆—O—Si(CH₃)₃
(CH₃)₃Si—[O—(CH₃)₂Si]₇—O—Si(CH₃)₃ (CH₃)₃Si—[O—(CH₃)₂Si]₈—O—Si(CH₃)₃
(CH₃)₃Si—[O—(CH₃)₂Si]₉—O—Si(CH₃)₃ (CH₃)₃Si—[O—(CH₃)₂Si]₁₀—O—Si(CH₃)₃
(CH₃)₃Si—[O—(CH₃)₂Si]₁₁—O—Si(CH₃)₃ (CH₃)₃Si—[O—(CH₃)₂Si]₁₂—O—Si(CH₃)₃
(CH₃)₃Si—[O—(CH₃)₂Si]₁₃—O—Si(CH₃)₃ (CH₃)₃Si—[O—(CH₃)₂Si]₁₄—O—Si(CH₃)₃
(CH₃)₃Si—[O—(CH₃)₂Si]₁₅—O—Si(CH₃)₃ (CH₃)₃Si—[O—(CH₃)₂Si]₁₆—O—Si(CH₃)₃
(CH₃)₃Si—[O—(CH₃)₂Si]₁₇—O—Si(CH₃)₃ (CH₃)₃Si—[O—(CH₃)₂Si]₁₈—O—Si(CH₃)₃
(CH₃)₃Si—[O—(CH₃)₂Si]₁₉—O—Si(CH₃)₃ (CH₃)₃Si—[O—(CH₃)₂Si]₂₀—O—Si(CH₃)₃
(CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂—O—Si(CH₃)₃ being particularly preferred.

Of course, mixtures of the above-mentioned silicones can also be contained in the agents as contemplated herein. Preferred silicones that can be used as contemplated herein have viscosities at about 20° C. of from about 0.2 to about 2 mm²s⁻¹, silicones having viscosities of from about 0.5 to about 1 mm²s⁻¹ being particularly preferred.

The hair treatment agents as contemplated herein may contain surfactant(s). In cleaning compositions (shampoos), in particular anionic surfactants have been found to be suitable and, in conditioning compositions, cationic surfactants are ingredients that are often used; owing to their advantageous properties, amphoteric surfactants are particularly preferably used in both shampoos and conditioners.

The hair treatment agents as contemplated herein may contain at least one anionic surfactant. Suitable anionic surfactants and emulsifiers for the compositions as contemplated herein include all anionic surface-active substances suitable for use on the human body.

These are exemplified by a water-solubilizing, anionic group such as a carboxylate, sulfate, sulfonate or phosphate group and a lipophilic alkyl group having approximately 8 to 30 C atoms. In addition, glycol or polyglycol ether groups, ester, ether and amide groups, and hydroxyl groups may be contained in the molecule.

Preferred hair treatment agents contain, based on their weight, from about 0.5 to about 20 wt. %, preferably from about 0.75 to about 15 wt. %, more preferably from about 1 to about 12 wt. %, and in particular from about 2 to about 10 wt. %, anionic surfactant(s).

Particularly preferred hair treatment agents as contemplated herein contain, based on their weight, from about 0.5 to about 20 wt. %, preferably from about 0.75 to about 15 wt. %, more preferably from about 1 to about 12 wt. %, and in particular from about 2 to about 10 wt. %, alkyl (ether) sulfates of general formula R—(OCH₂—CH₂)ₙ—OSO₃X, in which R is a straight-chain or branched, saturated or unsaturated alkyl group having 8 to 24 C atoms, n is the number 0 or from about 1 to about 12, and X is an alkali, alkaline-earth, ammonium or alkanolamine ion.

The hair treatment agents as contemplated herein may contain at least one amphoteric surfactant and/or at least one non-ionic surfactant.

Particularly preferred hair treatment agents as contemplated herein contain, based on their weight, from about 0.3 to about 20 wt. %, preferably from about 0.5 to about 8 wt. %, more preferably from about 0.75 to about 6 wt. %, and in particular from about 1 to about 5 wt. %, amphoteric surfactant(s).

Surfactants that have both a negatively-charged functional group and a positively-charged functional group are referred to as amphoteric surfactants or zwitterionic surfactants.

Particularly suitable zwitterionic surfactants are what are referred to as betaines, such as N-alkyl-N,N-dimethylammonium glycinates, for example cocoalkyl dimethylammonium glycinate, N-acylaminopropyl-N,N-dimethylammonium glycinates, for example cocoacylaminopropyl dimethylammonium glycinate, 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known by the INCI name cocamidopropyl betaine.

Further examples of suitable ampholytic surfactants are N-alkylglycines, N-alkylaminopropionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids, and alkylaminoacetic acids having in each case approximately 8 to 24 C atoms in the alkyl group. Particularly preferred ampholytic surfactants are N-cocoalkylaminopropionate, cocoacylaminoethyl aminopropionate, and C₁₂-C₁₈ acylsarcosine.

Particularly preferred hair treatment agents contain, as amphoteric surfactants, betaines of formula (Bet-I)

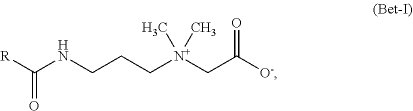

in which R represents a straight-chain or branched, saturated or mono- or polyunsaturated alkyl or alkenyl functional group having 8 to 24 carbon atoms.

In accordance with INCI nomenclature, these surfactants are referred to as amidopropyl betaines, the representatives derived from coconut fatty acids being preferred and referred to as cocamidopropyl betaines. It is particularly preferable as contemplated herein to use surfactants of formula (Bet-I) that are a mixture of the following representatives:

H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Particularly preferably, surfactants of formula (Bet-I) are used within narrower quantity ranges.

In this case, hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. %, surfactant(s) of formula (Bet-I) are preferred.

In addition to or instead of the ampho-surfactant(s) of formula (Bet-I), the hair treatment agents as contemplated herein may particularly preferably contain, as amphoteric surfactants, betaines of formula (Bet-II)

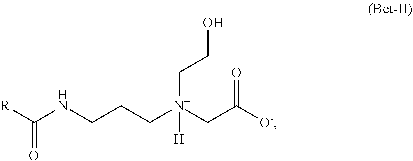

in which R represents a straight-chain or branched, saturated or mono- or polyunsaturated alkyl or alkenyl functional group having 8 to 24 carbon atoms.

In accordance with INCI nomenclature, these surfactants are referred to as amphoacetates, the representatives derived from coconut fatty acids being preferred and referred to as cocoamphoacetates.

For technical reasons relating to preparation thereof, surfactants of this type always also contain betaines of formula (Bet-IIa)

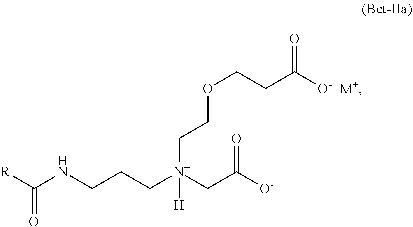

in which R represents a straight-chain or branched, saturated or mono- or polyunsaturated alkyl or alkenyl functional group having 8 to 24 carbon atoms, and M represents a cation.

In accordance with INCI nomenclature, these surfactants are referred to as amphodiacetates, the representatives derived from coconut fatty acids being preferred and referred to as cocoamphodiacetates.

It is particularly preferable as contemplated herein to use surfactants of formula (Bet-I) that are a mixture of the following representatives:

H₃C—(CH₂)₇—C(O)—NH—(CH₂)₂NH⁺(CH₂CH₂OH)CH₂CH₂COO⁻
H₃C—(CH₂)₉—C(O)—NH—(CH₂)₂NH⁺(CH₂CH₂OH)CH₂CH₂COO⁻
H₃C—(CH₂)₁₁—C(O)—NH—(CH₂)₂NH⁺(CH₂CH₂OH)CH₂CH₂COO⁻
H₃C—(CH₂)₁₃—C(O)—NH—(CH₂)₂NH⁺(CH₂CH₂OH)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂N⁺(CH₂CH₂OH)CH₂CH₂COO⁻

Particularly preferably, surfactants of the formula (Bet-II) are used within narrower quantity ranges. In this case, hair treatment agents as contemplated herein which contain, based on their weight, from about 0.25 to about 8 wt. %, more preferably from about 0.5 to about 7 wt. %, even more preferably from about 0.75 to about 6.5 wt. %, and in particular from about 1 to about 5.5 wt. %, surfactant(s) of formula (Bet-II) are preferred.

In summary, cosmetic agents as contemplated herein are preferred in which the functional group R in formulas (Bet-I) and (Bet-II) is selected from

H₃C—(CH₂)₇— H₃C—(CH₂)₉— H₃C—(CH₂)₁₁— H₃C—(CH₂)₁₃— H₃C—(CH₂)₁₅—
H₃C—(CH₂)7—CH═CH—(CH₂)₇— or mixtures thereof.

The hair treatment agents may contain non-ionic surfactant(s).

Hair treatment agents which are preferred as contemplated herein contain, based on their weight, from about 0.3 to about 10 wt. %, preferably from about 0.5 to about 8 wt. %, more preferably from about 0.75 to about 6 wt. %, and in particular from about 1 to about 5 wt. %, nonionic surfactant(s).

The hair treatment agents may contain cationic surfactant(s). As contemplated herein, it is possible to use cationic surfactants of the following types: quaternary ammonium compounds, esterquats, and amidoamines. Preferred quaternary ammonium compounds are ammonium halides, in particular chlorides and bromides, such as alkyl trimethylammonium chlorides, dialkyl dimethylammonium chlorides, and trialkyl methylammonium chlorides. The long alkyl chains of these surfactants preferably have 10 to 18 carbon atoms, such as in cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. Further preferred cationic surfactants are the imidazolium compounds known by the INCI names quaternium-27 and quaternium-83.

Particularly preferred hair treatment agents as contemplated herein contain as the cationic care substance, based on their weight, from about 0.05 to about 7.5 wt. %, preferably from about 0.1 to about 5 wt. %, particularly preferably from about 0.2 to about 3.5 wt. %, and in particular from about 0.25 to about 2.5 wt. %, cationic surfactant(s) from the group of quaternary ammonium compounds and/or esterquats and/or amidoamines.

Particularly preferred hair treatment agents as contemplated herein contain, based on the weight of the agent, from about 0.05 to about 20 wt. %, preferably from about 0.1 to about 10 wt. %, more preferably from about 0.25 to about 8 wt. %, and in particular from about 0.5 to about 7 wt. %, cationic surfactant(s), and preferably from about 0.05 wt. % to about 20 wt. %, more preferably from about 0.1 to about 10 wt. %, even more preferably from about 0.25 to about 8 wt. %, and in particular from about 0.5 to about 7 wt. %, behenyl trimethyl ammonium chloride.

The agents as contemplated herein may contain at least one cationic polymer.

Cationic polymers increase the nourishing performance of the hair treatment agents as contemplated herein (in particular the effectiveness of the agents as contemplated herein with respect to hair breakage).

Irrespective of which cationic polymer(s) is/are used, preferred hair treatment agents contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, cationic polymer(s).

Particularly preferably, cationic polysaccharide polymers are used as contemplated herein as the cationic polymers.

Cationic polysaccharide polymers increase the nourishing performance of the hair treatment agents as contemplated herein (in particular the effectiveness of the agents as contemplated herein with respect to hair breakage). Suitable cationic polysaccharide polymers may be selected from cationic cellulose compounds and/or from cationic guar derivatives.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of at least one polymer from the group of cationic cellulose polymers and/or cationic guar derivatives.

Cationic cellulose compounds within the meaning of the present disclosure are those that carry more than one permanent cationic charge in at least one side chain. Cellulose is constructed from beta-1,4-glycosidically linked D-glucopyranose units and forms unbranched, water-insoluble chains. A “side chain” of a cellulose is defined as chemical substituents which bond to the cellulose backbone and do not include only native cellulose, since they have been introduced subsequently, for example by chemical synthesis.

Quaternized cellulose polymers originating from hydroxy(C₂-C₄)alkyl celluloses, particularly preferably from hydroxyethyl celluloses, are preferred.

Polymers of this kind are known to a person skilled in the art and are commercially available from various companies. The cationic cellulose derivatives known by the INCI names polyquaternium-4, poly quaternium-10, poly quaternium-24, polyquaternium-67, and/or polyquaternium-72 are particularly preferred. Polyquaternium-10, polyquaternium-24, and/or polyquaternium-67 are very particularly preferred, with polyquaternium-10 being particularly preferred.

Preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of at least one polymer from the group of polyquaternium-4, polyquaternium-10, polyquaternium-24, polyquaternium-67, and/or polyquaternium-72.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular 0.15 to about 0.8 wt. %, polyquaternium-10.

Suitable cationic guar derivatives within the meaning of the present disclosure are cationic hydroxyalkyl guar derivatives, preferably cationic hydroxyethyl trimethylammonium guar and/or cationic hydroxypropyl trimethylammonium guar having average molecular weights between about 100,000 and about 2,000,000 daltons. The cationic guar polymers known by the INCI name guar hydroxypropyltrimonium chloride having a molecular weight (weight average) of between about 200,000 and about 1,600,000 daltons are particularly preferred. The cationic charge density of these guar polymers is preferably at least about 0.4 meq/g, preferably at least about 0.5 meq/g, and in particular at least about 0.6 meq/g. The nitrogen content thereof is preferably in the range of from about 1.1 to about 1.8 wt. % (based on the total weight thereof).

Cationic guar derivatives known by the INCI name guar hydroxypropyltrimonium chloride are known to a person skilled in the art and are obtainable from various providers under the trade names Cosmedia® Guar, N-Hance® and/or Jaguar®, for example.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, guar hydroxypropyltrimonium chloride.

In summary, hair treatment agents which are preferred as contemplated herein contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, cationic polymer(s), and preferably from about 0.01 wt. % to about 3 wt. %, more preferably from about 0.05 to about 2 wt. %, even more preferably from about 0.1 to about 1.5 wt. %, and in particular from about 0.15 to about 0.8 wt. %, of at least one polymer from the group of cationic cellulose polymers and/or cationic guar derivatives.

It has been found that alpha-substituted aldehydes can further enhance the effect of the agents as contemplated herein. Significant increases in performance are observed, in particular with regard to the inner structural strengthening of keratin fibers and the prevention of washout.

Hair treatment agents which are preferred as contemplated herein additionally contain, based on the weight of the agent, from about 0.001 to about 20 wt. % of at least one alpha-substituted aldehyde.

Preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, alpha-substituted aldehyde(s) of formula (I)

Y—CH(X)—CHO (I)

in which
X represents —OH, —Cl, —Br, —I, —O—(CH₂)ₙ—CH₃, where n=−1, 2, 3 or 4, or —O—(CH₂)ₘ—OH, where m=1, 2 or 3,
Y represents —H, —CH₃, H₃C—(CH₂)ₖ—, where k=1, 2, 3, 4, 5, 6, 7 or 8, —OH, —(CH₂)ₚ—OH, where p=1, 2, 3 or 4, or —CHO.

Aldehydes that are preferably to be used as contemplated herein are described on pages 36 to 38 of the priority document as numbers 1 to 176.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one alpha-substituted aldehyde from the group
2-hydroxypropanal (X=—OH, Y=—CH₃):
2-hydroxyhexanal (X=—OH, Y=H₃C—(CH₂)ₖ—, where k=3):
2-hydroxyoctanal (X=—OH, Y=H₃C—(CH₂)ₖ—, where k=5):
bromomalonaldehyde (X=Br, Y=—CH═):
2-(2-hydroxyethoxy)acetaldehyde (X=—O—(CH₂)ₚ—OH, where p=2, Y=—H):
glyceraldehyde (X=—OH, Y=—(CH₂)ₚ—OH, where p=1)

Complexing agents can further increase the effect of the agents as contemplated herein, complexing agents which are derived from polycarboxylic acids having been found to be particularly suitable.

Hair treatment agents which are preferred as contemplated herein contain, based on their weight, from about 0.001 to about 20 wt. % complexing agents from the group tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriaccetic acid (HEDTA).

The use of the three complexing agents mentioned first is very particularly preferred. Extremely preferred hair treatment agents as contemplated herein contain, based on their weight, from about 0.001 to about 20 wt. %, preferably from about 0.005 to about 15 wt. %, more preferably from about 0.01 to about 10 wt. %, even more preferably from about 0.05 to about 7.5 wt. %, and in particular from about 0.1 to about 5 wt. %, complexing agents from the group
a. tetrasodium-N,N-bis(carboxylatomethyl)-L-glutamate (tetrasodium glutamate diacetate, GLDA)

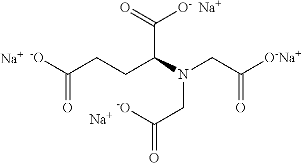

b. pentasodium diethylenetriaminepentaacetate (DTPA),

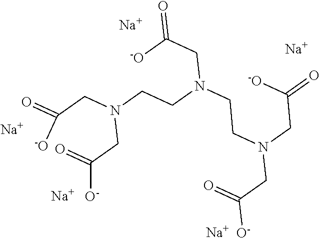

c. tetrasodium iminodisuccinate (IDS)

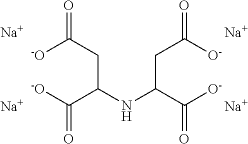

The agents as contemplated herein may contain only one of the three complexing agents mentioned. However, it is also possible for the agents as contemplated herein to contain two or all three of the above-mentioned complexing agents, the amount of all the complexing agents from the above-mentioned group contained in the agents being within an amount range of from about 0.01 to about 20 wt. %.

Hair treatment agents which are preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, tetrasodium-N,N-bis(carboxylatomethyl)-L-glutamate (tetrasodium glutamate diacetate, GLDA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, pentasodium diethylenetriaminepentaacetate (DTPA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, pentasodium diethylenetriaminepentaacetate (DTPA).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, pentasodium diethylenetriaminepentaacetate (DTPA), and from about 0.01 to about 10 wt. %, more preferably from about 0.05 to about 7.5 wt. %, and in particular from about 0.1 to about 5 wt. %, tetrasodium iminodisuccinate (IDS).

Hair treatment agents which are also preferred as contemplated herein contain, based on their weight, from about 0.1 to about 5 wt. % tetrasodium-N,N-bis(carboxylatomethyl)-L-glutamate (tetrasodium glutamate diacetate, GLDA), from about 0.1 to about 5 wt. % pentasodium diethylenetriaminepentaacetate (DTPA), and from about 0.1 to about 5 wt. % tetrasodium iminodisuccinate (IDS).

The hair treatment agents may contain at least one bivalent or trivalent metal salt. These lead to improved washout prevention. To achieve an optimum effect, it is advantageous for the metal salts to be present in dissolved form in the agents as contemplated herein. In a preferred embodiment, the hair cleaning and care agents as contemplated herein therefore contain water-soluble bivalent or trivalent metal salts. “Water-soluble” is understood here to mean that, at about 20° C., at least about 1 g of the salt in question can dissolve fully in about 1 L of water.

Suitable bivalent or trivalent metal salts may be selected from bivalent or trivalent organic and/or inorganic salts.

Particularly suitable cations within these salts may preferably be selected from alkaline-earth metal cations and from copper, zinc, iron(II), iron(III), and/or aluminum cations.

Alkaline-earth metal cations, and particularly preferably calcium and magnesium cations, are very particularly preferred. Particularly suitable organic anions within these salts may preferably be selected from formate, acetate, lactate, succinate, citrate, tartrate, malate, maleate, oxalate, and/or glycolate ions. Acetate, lactate, and/or citrate salts having the aforementioned cations are very particularly preferred.

Particularly preferred organic salts are calcium lactate, calcium citrate, calcium acetate, magnesium lactate, magnesium citrate, and/or magnesium acetate.

Particularly suitable inorganic anions within these salts may be selected from halide, sulfate, phosphate, and/or carbonate ions. Sulfate and/or halide ions such as chloride and bromide ions are very particularly preferred.

Particularly preferred inorganic salts are calcium chloride, calcium sulfate, magnesium chloride, and/or magnesium sulfate.

The weight proportion of the at least one bivalent or trivalent metal salt with respect to the total weight of the hair treatment agents as contemplated herein is preferably from about 0.01 to about 10 wt. %, preferably from about 0.1 to about 7.5 wt. %, more preferably from about 0.2 to about 5 wt. %, and in particular from about 0.3 to about 3 wt. %.

Hair treatment agents which are preferred as contemplated herein contain, based on their weight, from about 0.01 to about 10 wt. %, preferably from about 0.1 to about 7.5 wt. %, more preferably from about 0.2 to about 5 wt. %, and in particular from about 0.3 to about 3 wt. %, of at least one bivalent or trivalent metal salt from the group of organic or inorganic copper, zinc, iron(II), calcium, magnesium, iron(III), and/or aluminum salts.

Water-soluble salts are particularly preferred in this embodiment. Calcium lactate, calcium citrate, calcium acetate, magnesium lactate, magnesium citrate, magnesium acetate, calcium halides, calcium hydroxide, magnesium halides, and/or magnesium hydroxide are very particularly preferred in this embodiment.

The hair treatment agents contain the above-described active ingredients preferably in a cosmetically acceptable carrier. Within the context of the present disclosure, this is understood to preferably mean an aqueous or aqueous-alcoholic carrier.

The cosmetic carrier preferably contains at least about 50 wt. %, more preferably at least about 60 wt. %, particularly preferably at least about 70 wt. %, and more particularly preferably at least about 75 wt. %, water.

Furthermore, the cosmetic carrier may contain from about 0.01 to about 40 wt. %, preferably from about 0.05 to about 30 wt. %, and in particular from about 0.1 to about 20 wt. %, of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of said alcohols.

Water-soluble alcohols are particularly preferred. Ethanol, 1,2-propylene glycol, glycerol, benzyl alcohol, and mixtures of said alcohols are particularly preferred.

It is advantageous for very good (scalp) skin compatibility of the hair treatment agents as contemplated herein if said agents have a slightly acidic pH.

It has been found that the agents as contemplated herein have particularly good skin compatibility and mildness in a pH range of from about 4.2 to about 5.8.

In a first preferred embodiment, the hair treatment agents as contemplated herein therefore preferably have a pH in the range of from about 4.2 to about 5.8, more preferably from about 4.3 to about 5.6, particularly preferably from about 4.4 to about 5.5, extremely preferably from about 4.5 to about 5.4, and particularly preferably from about 4.7 to about 5.3.

The hair treatment agents as contemplated herein may contain vegetable oils, vegetable butters, and/or vegetable waxes. These vegetable oil components give the hair improved combability and stylability and increase hair shine.

Suitable vegetable oil components include natural (vegetable) oils and/or butters which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Carnauba wax, beeswax, and/or candelilla wax can preferably be used as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter. Apricot kernel oil, argan oil, olive oil, and/or jojoba oil are particularly preferred.

In a preferred embodiment, the hair treatment agents as contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is particularly preferable if the hair treatment agents as contemplated herein contain (sweet) almond oil, peach kernel oil, apricot kernel oil, amaranth seed oil, argan oil, olive oil, jojoba oil, cocoa butter, and/or shea butter.

The weight proportion of the at least one vegetable oil, vegetable butter, and/or vegetable wax with respect to the total weight of the hair treatment agents as contemplated herein is preferably from about 0.02 to about 2.50 wt. %, more preferably from about 0.03 to about 2.00 wt. %, particularly preferably from about 0.04 to about 1.50 wt. %, and in particular from about 0.05 to about 1.00 wt. %.

In addition to the aforementioned essential and optional components, the hair treatment agents as contemplated herein may contain, in a further preferred embodiment for further increasing the nourishing properties of the agents, at least one further active hair-conditioning ingredient, which can be selected from the group of
vitamins,
plant extracts, and/or
glycerol.

The weight proportion of the vitamin(s), vitamin derivative(s), and/or the vitamin precursor(s) with respect to the total weight of the hair treatment agents is preferably from about 0.001 to about 2 wt. %, particularly preferably from about 0.005 to about 1 wt. %, and in particular from about 0.01 to about 0.5 wt. %.

Suitable plant extracts are to be understood to mean extracts that can be prepared from all the parts of a plant.

The plant extracts can be used in the hair treatment agents as contemplated herein (based on the total weight of the agents) preferably in an amount of from about 0.01 to about 10 wt. %, more preferably from about 0.05 to about 7.5 wt. %, and in particular from about 0.1 to about 5 wt. %.

Glycerol can be added to the hair cleaning and care agents separately in an amount of up to about 10 wt. % (based on the total weight of the agent). However, it may also be a component of the above-mentioned aqueous-alcoholic carrier.

It has been found that the hair treatment agents as contemplated herein are also suitable for use as an anti-dandruff preparation.

The total weight of anti-dandruff agents with respect to the total weight of the hair treatment agents can be preferably from about 0.01 to about 10 wt. %, more preferably from about 0.025 to about 7.5 wt. %, particularly preferably from about 0.05 to about 5 wt. %, and in particular from about 0.075 to about 3 wt. %.

Suitable anti-dandruff active ingredients can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

Other active substances, auxiliary substances and additives that can be contained in the hair treatment agents as contemplated herein are, for example:
humectants,
perfume,
UV filters,
thickening agents such as gelatins or plant gums, for example agar-agar, guar gum, alginates, xanthan gum, gum arabic, karaya gum, locust bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays and sheet silicates, such as bentonite or fully synthetic hydrocolloids such as polyvinyl alcohol, the Ca, Mg or Zn soaps,
structurants such as maleic acid and lactic acid,
dimethyl isosorbide,
cyclodextrins,
fiber-structure-improving active ingredients, in particular mono-, di-, and oligosaccharides such as, for example, glucose, galactose, fructose, fruit sugar, and lactose,
dyes for coloring the agent,
active ingredients such as bisabolol and/or allantoin,
complexing agents such as EDTA, NTA, β-alanine diacetic acid and phosphonic acids,
ceramides. Ceramides are understood to mean N-acylsphingosine (fatty acid amides of sphingosine) or synthetic analogs of such lipids (so-called pseudo-ceramides),
propellants such as propane-butane mixtures, N₂O, dimethyl ether, CO₂ and air,
antioxidants,
additional viscosity regulators, such as salts (NaCl).

The agents as contemplated herein can be formulated as what are referred to as rinse-off products, i.e. are rinsed out of the hair again after a specific contact time. This contact time is preferably less than an hour, i.e. the user preferably does not leave the products in the hair until the next time the hair is washed.

The present disclosure therefore further relates to a method for treating hair in which an agent as contemplated herein is applied to dry or wet hair, is left there for a period of from about 10 to about 300 seconds, and is subsequently rinsed out.

The agents as contemplated herein can also be formulated as what are referred to as leave-on products, i.e. are not rinsed out of the hair, but are instead left there until the next time the hair is washed.

The present disclosure therefore further relates to a method for treating hair in which an agent as contemplated herein is applied to dry or wet hair and is left there until the next time the hair is washed.

The agents as contemplated herein lead to a significantly increased strengthening of the internal and external hair structure. A further subject of the present disclosure is therefore the use of agents as contemplated herein for strengthening the hair structure, in particular the internal hair structure.

Within the meaning of the present disclosure, “structure strengthening” is understood to mean a reduction in the damage to keratin fibers caused by a wide range of influences. In this case, re-establishing the natural strength plays a significant role, for example. Restructured fibers are exemplified, for example, by improved shine, by an improved feel, and by easier combability. In addition, they have optimized strength and elasticity. Successful structure strengthening or restructuring can be verified physically as an increase in the melting point in comparison with damaged fibers.

The agents as contemplated herein also lead to considerably increased stability of artificial colors against the washout of color. Chemically colored hair can thus be washed much more frequently using the agents as contemplated herein, without this leading to undesired bleeding or fading of the color.

The present disclosure therefore further relates to the use of agents as contemplated herein for reducing the washout of color from chemically colored hair.

The statements made about the agents as contemplated herein apply, mutatis mutandis, to particularly preferred embodiments of the methods as contemplated herein and of the uses as contemplated herein.

### EXAMPLES

All values in wt. %.
Succinimidyl esters used:

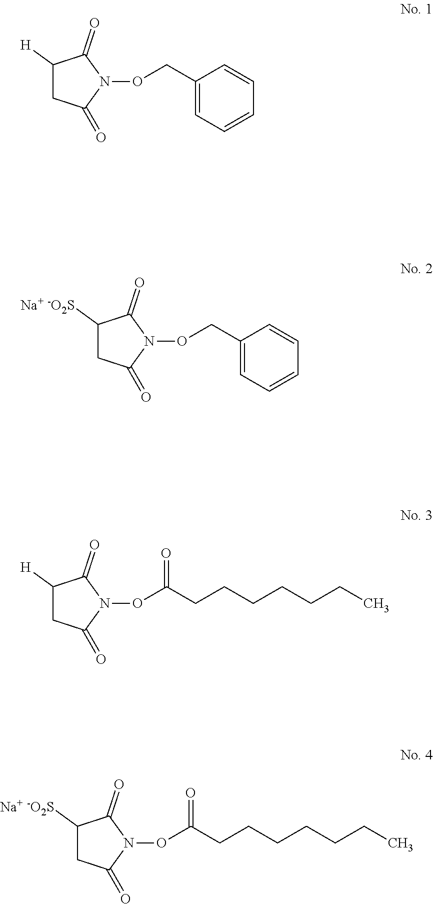

Hair shampoo:

| | 1 | 2 | 3 | 4 | 5 | 6 |
|---|---|---|---|---|---|---|
| Sodium laureth sulfate | 10 | 10 | 10 | 10 | 10 | 10 |
| Cocoamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| | | — | — | — | | — |
| Succinimidyl ester no. 1 | 0.1 | | — | — | 0.1 | — |
| | — | | | | — | |
| Succinimidyl ester no. 2 | — | 0.15 | — | — | — | — |
| Succinimidyl ester no. 3 | — | — | 0.1 | | — | 0.1 |
| | | | — | | | — |
| Succinimidyl ester no. 4 | — | — | — | 0.15 | — | — |
| Glyceraldehyde | — | — | — | — | 0.1 | |
| | | | | | — | |
| Bromomalonaldehyde | | | | | | 0.2 |
| Panthenol | 0.5 | 0.5 | 0.5 | 0.5 | 0.5 | 0.5 |
| 2-hydroxyoctanal | 1.0 | 1.0 | 1.0 | 1.0 | 1.0 | 1.0 |
| Hydrolyzed wheat protein | 1.0 | 2.0 | 3.5 | 1.0 | 0.8 | 1.0 |
| Laureth-2 | 1.2 | 1.2 | 1.2 | 1.2 | 1.2 | 1.2 |
| Sodium chloride | 1.3 | 1.0 | 1.0 | 1.2 | 1.1 | 1.3 |
| Water, preservative, perfume | up to | up to | up to | up to | up to | up to |
| oils | 100 | 100 | 100 | 100 | 100 | 100 |

Hair conditioner:

| | 1 | 2 | 3 | 4 | 5 | 6 |
|---|---|---|---|---|---|---|
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar hydroxypropyl- | 0.2 | 0.2 | 0.2 | 0.2 | 0.2 | 0.2 |
| trimonium chloride | | | | | | |
| Polyquaternium-37 | 0.4 | 0.4 | 0.4 | 0.4 | 0.4 | 0.4 |
| Hydrolyzed keratin | 0.1 | 0.1 | 0.1 | 0.1 | 0.1 | 0.1 |
| Shea butter | 1.5 | 1.5 | 1.5 | 1.5 | 1.5 | 1.5 |
| Behenoyl | 1.5 | 1.5 | 1.5 | 1.5 | 1.5 | 1.5 |
| PG-trimoniumchloride | | | | | | |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Lactic acid | 0.1 | 0.1 | 0.1 | 0.1 | 0.1 | 0.1 |
| Succinimidyl ester no. 1 | 0.1 | — | — | — | — | — |
| Succinimidyl ester no. 2 | — | 0.15 | — | — | 0.1 | — |
| Succinimidyl ester no. 3 | — | — | 0.1 | — | — | — |
| Succinimidyl ester no. 4 | — | — | — | 0.15 | — | 0.1 |
| 2-hydroxyhexanal | — | — | — | — | 0.1 | — |
| 2-hydroxyoctanal | — | — | — | — | — | 0.2 |
| Amodimethicone | 0.8 | 0.8 | 0.8 | 0.8 | 0.8 | 0.8 |
| Water, preservative, perfume | up to | up to | up to | up to | up to | up to |
| oils | 100 | 100 | 100 | 100 | 100 | 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent comprising, based on the weight of the hair treatment agent,
a) from about 0.001 to about 10 wt. % of at least one succinimidyl ester, and
b) from about 0.001 to about 20 wt. % of at least one protein hydrolyzate.

2. The hair treatment agent according to claim 1, wherein the hair treatment agent comprises, based on the weight of the hair treatment agent, from about 0.01 to about 10 wt. % succinimidyl ester(s) of formula (I) 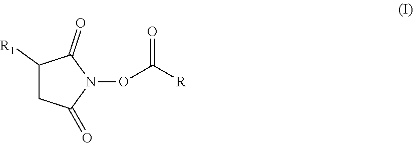
in which
R1 represents —H or an ionic group, and
R represents an optionally substituted, saturated or unsaturated, linear, branched or cyclic, aliphatic or aromatic hydrocarbon functional group having at least 5 carbon atoms.

3. The hair treatment agent according to claim 1, wherein the hair treatment agent comprises, based on the weight of the hair treatment agent, from about 0.01 to about 10 wt. %, of at least one succinimidyl ester having a formula from the group of formula (Ia), (Ib), (Ic), and (Id): 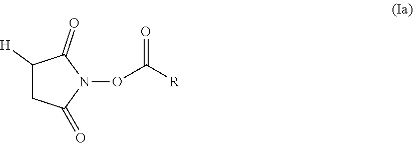
in which R represents —Ph or —(CH₂)nCH₃, where n=4, 5, 6, 7, 8, 9 or 10, 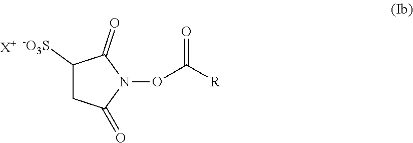
in which R represents —Ph or —(CH₂)nCH₃, where n=4, 5, 6, 7, 8, 9 or 10, and X represents H, a monovalent cation, or the n-th part of an n-valent cation,

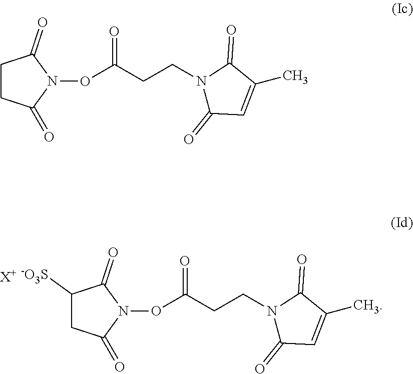

4. The hair treatment agent according to claim 1, wherein the hair treatment agent comprises, based on the weight of the hair treatment agent, from about 0.01 to about 15 wt. %, protein hydrolyzate(s) from the group of elastin, collagen, keratin, silk, and milk protein hydrolyzate, soybean, almond, pea, moringa, potato, and wheat protein hydrolyzate.

5. The hair treatment agent according to claim 1, wherein the hair treatment agent comprises at least one oligopeptide having an amino acid sequence Tyr-Glu-Glu-Glu-Ile 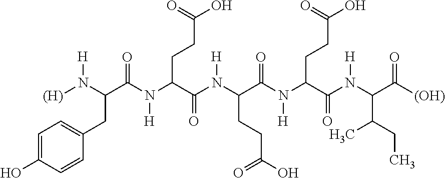
wherein the amino group is present in a free or protonated form and wherein the carboxy groups are present in a free or deprotonated form.

6. The hair treatment agent according to claim 1, wherein the hair treatment agent comprises, based on the weight of the hair treatment agent, from about 0.3 to about 10 wt. %, amphoteric surfactant(s).

7. The hair treatment agent according to claim 1, wherein the hair treatment agent comprises, based on the weight of the hair treatment agent, from about 0.01 to about 3 wt. % of at least one polymer from the group of cationic cellulose polymers and/or cationic guar derivatives.

8. The hair treatment agent according claim 1, wherein the hair treatment agent comprises, based on the weight of the hair treatment agent, from about 0.05 to about 20 wt. %, behenyl trimethyl ammonium chloride.

9. The hair treatment agent according to claim 1, wherein the hair treatment agent further comprises, based on the weight of the hair treatment agent, from about 0.001 to about 20 wt. % of at least one alpha-substituted aldehyde.

10. The hair treatment agent according to claim 1, wherein the hair treatment agent comprises, based on the weight of the hair treatment agent, from about 0.01 to about 10 wt. %, alpha-substituted aldehyde(s) of formula (III)
Y—CH(X)—CHO (III)
in which
X represents —OH, —Cl, —Br, —I, —O—(CH₂)ₙ, where n=1, 2, 3 or 4, or —O—(CH₂)ₘ—OH, where m=1, 2 or 3, and
Y represents —H, —CH₃, H₃C—(CH₂)ₖ—, where k=1, 2, 3, 4, 5, 6, 7 or 8, —OH, or —(CH₂)ₚ—OH, where p=1, 2, 3 or 4, or —CHO.

11. The hair treatment agent according to claim 1, wherein the hair treatment agent comprises, based on the weight of the hair treatment agent, from about 0.01 to about 10 wt. % of at least one alpha-substituted aldehyde from the group of:
2-hydroxypropanal (X=—OH, Y=—CH₃): 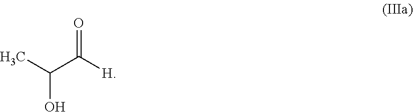
2-hydroxyhexanal (X=—OH, Y=H₃C—(CH₂)ₖ—, where k=3): 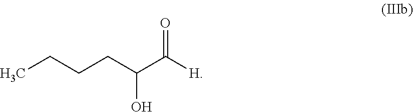
2-hydroxyoctanal (X=—OH, Y=H₃C—(CH₂)ₖ—, where k=5): 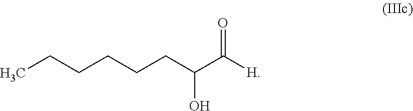
bromomalonaldehyde (X=Br, Y=—CH═): 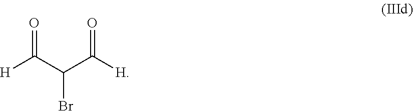
2-(2-hydroxyethoxy)acetaldehyde (X=—O—(CH₂)ₚ—OH, where p=2, Y=—H): 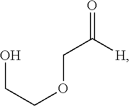
and
glyceraldehyde (X=—OH, Y=—(CH₂)ₚ—OH, where p=1)

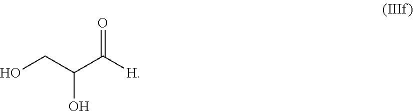

12. The hair treatment agent according to claim 1, wherein the hair treatment agent comprises, based on the weight of the hair treatment agent, from about 0.001 to about 20 wt. % complexing agents from the group of tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriaccetic acid (HEDTA).

13. The hair treatment agent according to claim 1, wherein the hair treatment agent comprises, based on the weight of the hair treatment agent, from about 0.001 to about 20 wt. % complexing agents from the group
a. tetrasodium-N,N-bis(carboxylatomethyl)-L-glutamate (tetrasodium glutamate diacetate, GLDA) 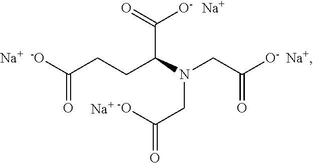
b. pentasodium diethylenetriaminepentaacetate (DTPA), 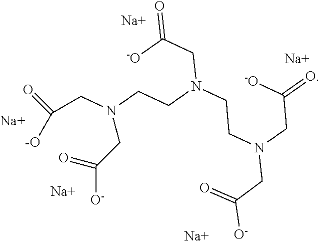
and
c. tetrasodium iminodisuccinate (IDS)

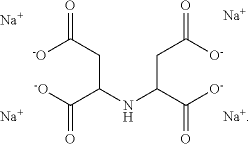

14. A method for treating hair comprising:
applying a hair treatment agent to dry or wet hair, wherein the hair treatment agent comprises, based on the weight of the hair treatment agent, from about 0.001 to about 10 wt. % of at least one succinimidyl ester, and from about 0.001 to about 20 wt. % of at least one protein hydrolyzate,
leaving the hair treatment agent on the hair for a selected period of time, and
subsequently rinsing the hair treatment agent from the hair.

15. The method of claim 14, wherein the selected period of time is from about 10 to about 300 seconds.
